(19) United States
(12) Patent Application Publication
(10) Pub. No.: US 2013/0061858 A1
(43) Pub. Date: Mar. 14, 2013

(54) UNDERGARMENT APPARATUS FOR GROIN COMFROT AND RELIEF

(71) Applicants: Sou Fou Saeteurn, Oakland, CA (US); Steven Tsing, Hayward, CA (US); Choy Tuyen, San Leandro, CA (US); Simon Louie, San Francisco, CA (US)

(72) Inventors: Sou Fou Saeteurn, Oakland, CA (US); Steven Tsing, Hayward, CA (US); Choy Tuyen, San Leandro, CA (US); Simon Louie, San Francisco, CA (US)

(21) Appl. No.: 13/039,234

(22) Filed: Mar. 2, 2011

Related U.S. Application Data
(60) Provisional application No. 61/309,647, filed on Mar. 2, 2010.

(51) Int. Cl. A61F 13/00

(52) U.S. Cl. 128/891

(57) ABSTRACT

This application describes an apparatus providing groin relief, which comprises, an undergarment which comprises a brief portion having an opening to allow male groin anatomy to extend through, and a boxer portion. The brief portion is inserted into the boxer portion so that the outer edges of the brief portion and the inner edges of the boxer portion are aligned, the brief portion is generally elastic, and an edge along the opening of the brief portion comprises elastic trim.

### BACKGROUND

This application relates generally to apparatus and method for undergarment and, more specifically, to a new undergarment for male groin comfort and relief. This application is a non-provisional application replacing the provisional application No. 61/309,647, titled “MALE BOXER SHORTS INTEGRATED TO MALE BOXER BRIEFS WITH FRONTAL GROIN RELIEF AREA OPEN TO A SINGLE WAIST BAND.”

Conventional products available on the marketplace provide compromised solutions to male undergarment. Conventional briefs are elastic thus provide protections from chafing or rubbing the skin of the inner thigh against the sensitive groin area by providing one or more than one layer of fabric covering the sensitive groin area and the inner thigh thereby separating the male scrotum and the inner thighs. However, such design holds testicle and scrotum close to the body and does not allow the natural male anatomical and physiological changes such as testicles' movements relative to the body to adjust for temperature changes. Conventional boxers, on the other hand, allow for testicles and scrotum to hang thereby allowing testicular movements relative to body to adjust for temperature changes, and provide comfort. However, such design results in scrotum to come in contact and rub against the skin of the inner thigh, and if the wearer is in high temperature environment, the rubbing and contact against inner thigh can create excessive sweat, chafing, and odor. Boxer also has a tendency to ride up and causes excessive urine to come in contact with the skin of their thighs after urination.

### SUMMARY

This application describes an apparatus providing groin relief, which comprises, an undergarment which comprises a brief portion having an opening to allow male groin anatomy to extend through, and a boxer portion. The brief portion is inserted into the boxer portion so that the outer edges of the brief portion and the inner edges of the boxer portion are aligned, the brief portion is generally elastic, and an edge along the opening of the brief portion comprises elastic trim.

### BRIEF DESCRIPTION OF THE DRAWINGS

The drawings illustrate the design and utility of various embodiments of the present application, in which similar elements are referred to by common reference numerals. In order to better appreciate how advantages and objects of the present application are obtained, a more particular description will be rendered by reference to specific embodiments thereof, which are illustrated in the accompanying drawings. Understanding that these drawings depict only typical embodiments and are not therefore to be considered limiting of its scope. The present application will be described and explained with additional specificity and detail through the use of the accompanying drawings in which:

FIG. 1 is a schematic illustration of the frontal view of the brief portion of the undergarment without frontal coverage of the groin area of the male anatomy;

FIG. 2 is a schematic illustration of the male anatomy of the wearer relative to the brief portion of the undergarment in accordance to some embodiments of this application;

FIG. 3 is a schematic illustration of the boxer portion in accordance to some embodiments of this application;

FIG. 4 is a schematic illustration of the integrated brief portion and boxer portion in accordance to some embodiments of this application;

FIG. 5 is a schematic illustration of the rear view of the integrated brief portion and boxer portion in accordance to some embodiments of this application.

FIG. 6 is a schematic illustration of one of the embodiments to construction the brief portion in accordance to some embodiments of this application.

### DETAILED DESCRIPTION

Various embodiments of the present application are described hereinafter with reference to the figures. It should be noted that the figures are not drawn to scale and elements of similar structures or functions are represented by like reference numerals throughout the figures. It should also be noted that the figures are only intended to facilitate the description of specific embodiments. They are not intended as an exhaustive description of the present application or as a limitation on the scope of the present application. In addition, an aspect or a feature described in conjunction with a particular embodiment is not necessarily limited to that embodiment and can be practiced in any other embodiments of the present application.

This application provides for an apparatus and a method for an undergarment to provide comfort and protection to wearer. The undergarment combines advantages of brief as well as boxer, and allow the male anatomy to have physiological movement adjustable by temperature, and to protect the groin sensitive area from abrasion and chafing.

The embodiments of apparatus and/or method comprise: 1) brief portion: this portion of undergarment is at least partially elastic and generally covers inner thigh of the wearer. The waist band is elastic to provide retention of the brief portion to the wearer's waist. Elastic fabric extends from the waist band down to cover outer and inner thigh. The front groin section, or a triangular shaped portion, of the brief portion is removed or removable to allow the male or female groin anatomical feature to be exposed and visible beyond the coverage of the brief portion. The rear of the brief portion provides coverage to the buttock of the wearer. The brief portion may be pieced together by several pieces of fabric; 2) boxer portion: this portion of undergarment may be partially elastic and generally covers the wearer from waist down to the thighs. The shape of the boxer portion resembles a pair of shorts but the length may vary from as long as knee-length to as short as upper thigh-length. The waist band is elastic to provide retention of the boxer portion to the wearer's waist. The frontal groin section is covered and optionally includes an opening to allow exposure of the groin anatomy. The rear of the boxer portion provides coverage to the buttock of the wearer. The boxer portion may be pieced together by several pieces of fabric; 3) integration: this portion of the undergarment integrates the brief portion and the boxer portion. The brief portion and boxer portion are at least partially integrated at the waist section. The brief portion and the boxer portion may also be integrated at sections other than the waist section. The integration may be performed by sewing or stitching via threads of various materials. Glue or other adhesives may also be used.

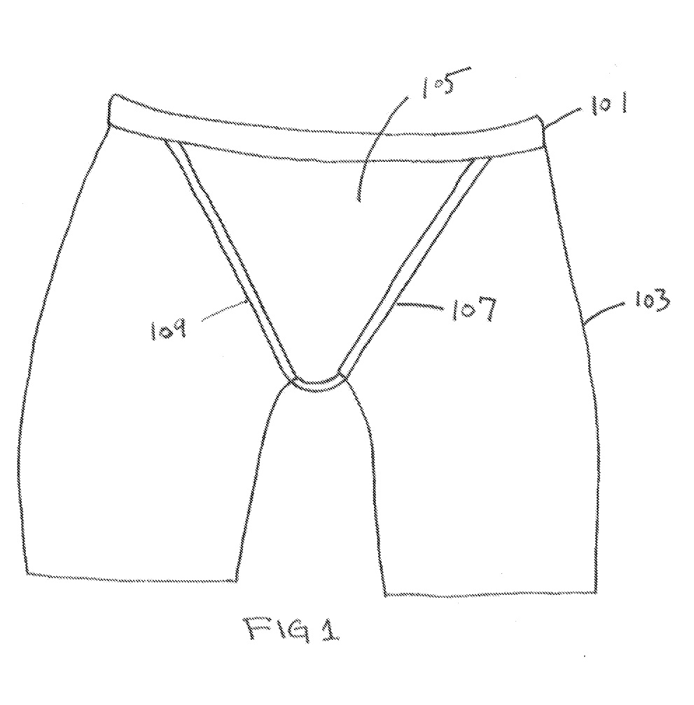

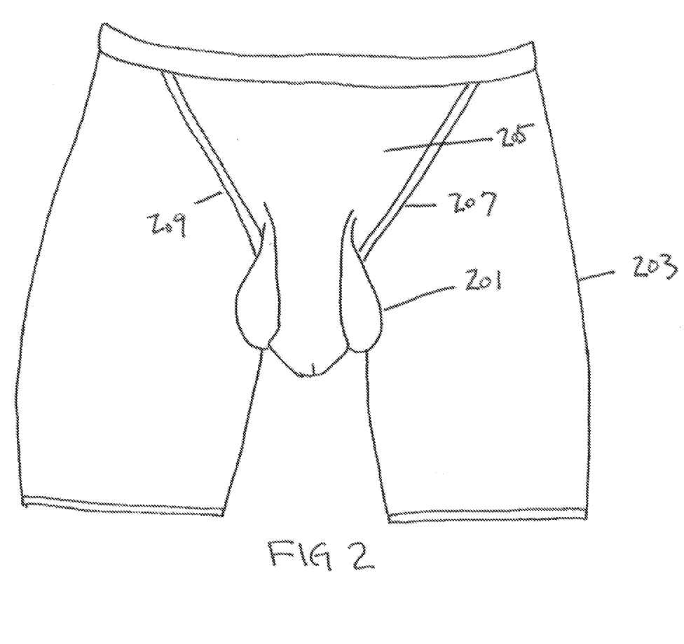

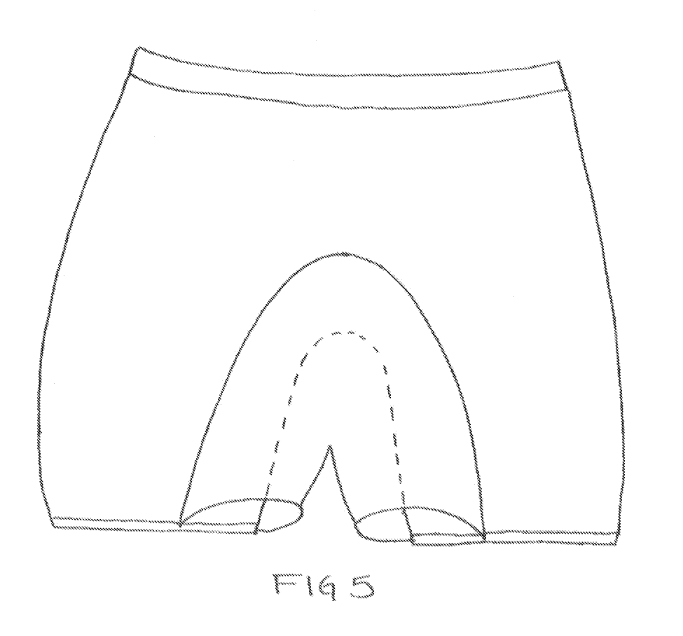

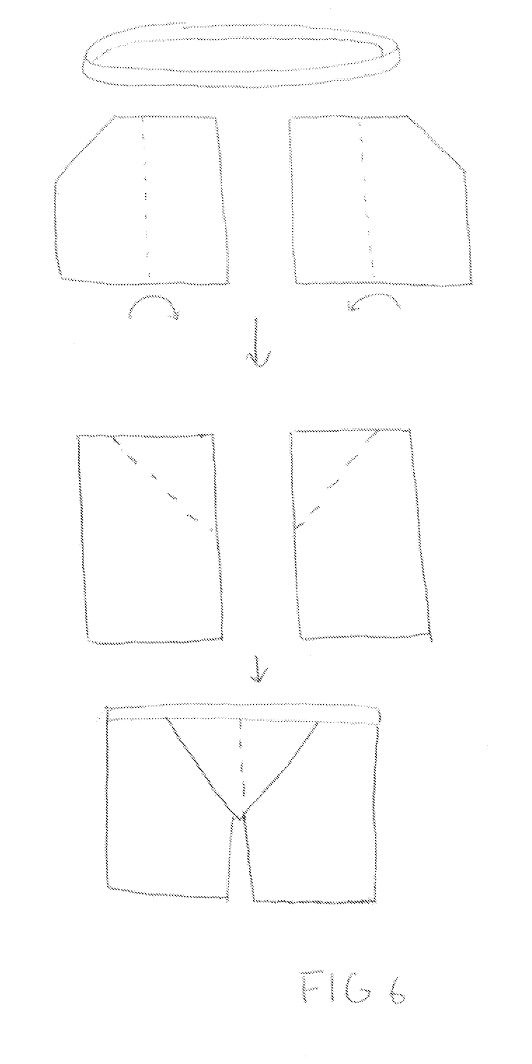

In one embodiment (shown in FIG. 1), the frontal view of the brief portion of the undergarment is depicted. The waist band 101, the thigh coverage 103, the groin opening 105, and the groin opening trims 107 and 109 generally comprise the brief portion. The waist band 101, the thigh coverage 103, and the groin opening trims 107 and 109 are optionally elastic and flexible. Each component can be made of varieties of fabrics, optionally elastic, including cotton, nylon, synthetic fiber, or in any combination thereof. Each component can be made in different lengths or sizes to fit wearer of different sizes. Each component may also be made in fabrics and materials that absorbs liquid (e.g., sweat) in order to provide further comfort. The rear view of the brief portion of the undergarment is depicted (shown in FIG. 5). The wearer's anatomy 201 is shown in FIG. 2 depicting the relative relationship of the wearer's anatomy 201 to the brief portion of the undergarment and the groin opening 205. As shown, the anatomy 201 is exposed outside of the brief portion, and the anatomy 201 is shielded from the inner thigh of the wearer by the thigh coverage 203 and bordered by groin opening trims 207 and 209. Note that groin opening 105 or 205 can be as large as an opening extends all the way to the waist band, or an opening that only allows the wearer's anatomy to extend through so as the groin opening encircles the base of the wearer's anatomy. Also note that the brief portion of the undergarment may be pieced together by several pieces of fabric in order to achieve the desirable shape and for the ease of manufacturing. For example, if the brief portion is made by piecing together three pieces of fabric: waist band (a circular, thin piece of fabric), two equally shaped pieces of fabric such that each looks like a square with one triangular shaped fabric removed (“thigh coverage piece”) from one corner where each comprises the thigh coverage 203, one skilled in the art would understand that assembling the brief portion entails the following: 1) each of the thigh coverage piece is folded in half, where the missing corner is facing each other; 2) the longer edge of the two thigh coverage pieces are sewn together partially, which would be the seam at the rear of the brief portion; 3) the remaining unsown part of the longer edge is sewn together with the folded edge; and 4) the waist band is sewn with the folded edge where the missing corner resides (see FIG. 6 for demonstration). Applicants submit that there are many ways to construct the brief portion. The aforementioned steps are just one example and the construction thereof is not limited to this particular example.

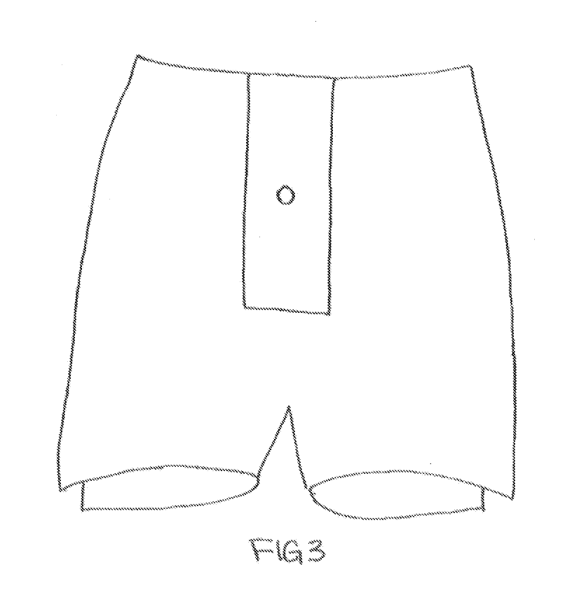

In another embodiment (shown in FIG. 3), the frontal view of the boxer portion of the undergarment is depicted. The boxer portion comprises waist band for retention on the wearer, a front panel and a rear panel wherein the two panels are integrated on the sides and the groin section along the edges, and two openings are allowed for the wearer's legs to extend through. The front panel of the boxer portion optionally contains an enclosable opening for ease of letting the wearer's anatomy to extend through for ease of urination. Note that the boxer portion may be constructed according to the example demonstrated in FIG. 6, but similarly, Applicants submit that the method of construction is not limited to this example.

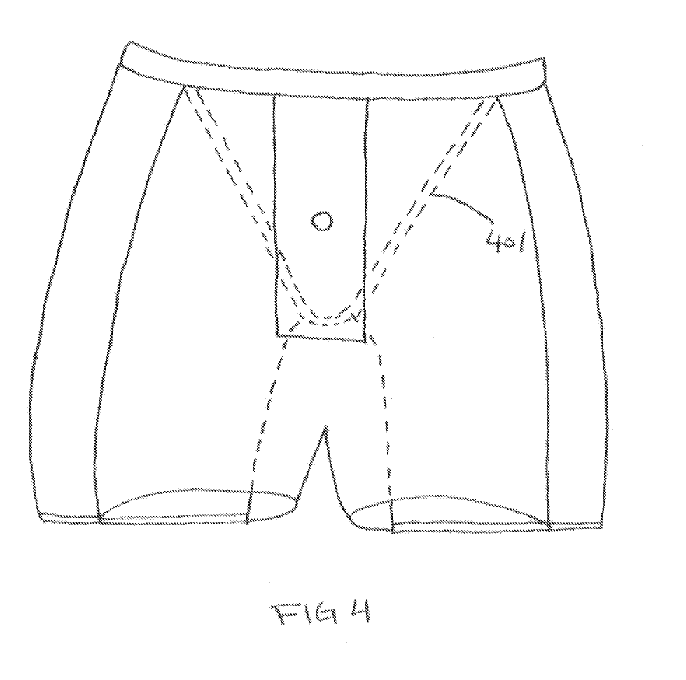

Referring to FIG. 4, the brief portion and the boxer portion are integrated to form the undergarment. The brief portion is inserted into the interior of the boxer portion, where the thigh coverings of the brief portion are extended down the boxer portion's aforementioned openings. As shown, the dotted lines 401 represent the brief portion being inserted into/integrated with the boxer portion and the relative position of the two portions. The two portions are at least integrated by binding the waist bands of the two portions. Two portions may optionally be additionally bound along the outer edge of the brief portion to the interior lining of the boxer portion. The binding may be done by sewing (in single, double, pattern stitches, and with materials such as cotton threads, synthetic threads, wood threads), buttoning (placing buttons along the edges and allow the outer edge of the brief portion to be buttoned to the inner lining of the boxer portion), zipping (placing zipper along the outer edge of the brief portion to be zipped to the inner lining of the boxer portion). The binding may also be optional as the wearer may choose to wear the brief portion and the boxer portion together but not integrated/unbound. The binding may be optional to allow the wearer to clean the brief portion and the boxer portion separately.

FIG. 5 shows the rear view of the undergarment where the brief portion and the boxer portion are integrated.

As understood by those skilled in the art, the fabrics and materials may be of different colors and patterns to manufacture both portions of the undergarment.

The undergarment provides a wearer a method of relief and comfort. The wearer, by wearing the undergarment, allows the anatomy to extend through the groin opening provided by the brief portion. The anatomy thereby may freely hang and rise or drop relative to the body of the wearer in responding to the temperature of the surrounding. The wearer's anatomy is hidden by the boxer portion as outer layer of the undergarment to cover the exposed anatomy. Wearing the undergarment (i.e., both the brief portion and the boxer portion) provides both comfort and protection from chafing and heat.

There are many possible configurations to the apparatus described in this application. Those skilled in the art will appreciate that various other modifications may be made within the spirit and scope of the invention. All these or other variations and modifications are contemplated by the inventors and within the scope of the invention.

What is claimed:

1. An apparatus to provide groin relief, comprising:
an undergarment comprising:
a brief portion having an opening to allow male groin anatomy to extend through;
a boxer portion;
wherein the brief portion is inserted into the boxer portion so that the outer edges of the brief portion and the inner edges of the boxer portion are aligned;
wherein the brief portion is generally elastic; and
wherein an edge along the opening of the brief portion comprises elastic trim.

2. The apparatus of claim 1, wherein the brief portion further comprises a front panel and a rear panel, the front panel having the opening.

3. The apparatus of claim 1, wherein the boxer portion optionally having an enclosable opening.

4. The apparatus of claim 1, wherein the brief portion and the boxer portion are generally integrated along an upper edge.

5. The apparatus of claim 4, wherein the brief portion and the boxer portion are generally integrated along all edges.

6. The apparatus of claim 4, wherein the male groin anatomy is at least partially supported by the opening of the brief portion.

7. A method to provide groin relief to a user, comprising:
wearing an undergarment comprising:
a brief portion having an opening and a boxer portion, wherein the brief portion is inserted into the boxer portion so that the outer edges of the brief portion and the inner edges of the boxer portion are aligned, the brief portion is generally elastic; and an edge along the opening of the brief portion comprises elastic trim;
extending male groin anatomy through the opening; and
protecting male groin anatomy from friction against inner thigh.
